(19) United States
(12) Patent Application Publication
(10) Pub. No.: US 2024/0057913 A1
(43) Pub. Date: Feb. 22, 2024

(54) DEVICE FOR ANALYSING A VEHICLE DRIVER'S PERCEPTION OF A DANGER AND ASSOCIATED METHOD

(71) Applicant: VALEO COMFORT AND DRIVING ASSISTANCE, Créteil (FR)

(72) Inventors: Stephanie Degrand, Creteil (FR); Laora Kerautret, Creteil (FR); Jordan Navarro, Lyon (FR)

(73) Assignees: VALEO COMFORT AND DRIVING ASSISTANCE, Créteil (FR); VALEO COMFORT AND DRIVING ASSISTANCE, Créteil (FR)

(21) Appl. No.: 18/270,341

(22) PCT Filed: Dec. 29, 2021
(86) PCT No.: PCT/FR2021/052465

§ 371 Date: Jun. 29, 2023

(30) Foreign Application Priority Data
Dec. 31, 2020 (FR) 2014293

(51) Int. Cl. A61B 5/16; A61B 5/0205; A61B 5/00; A61B 5/117; A61B 5/18

(52) U.S. Cl. CPC A61B 5/165; A61B 5/0205; A61B 5/6893; A61B 5/117; A61B 5/18; A61B 5/163; A61B 2503/22; A61B 5/024

(57) ABSTRACT

A device (1) for analysing a vehicle driver's perception of a danger comprises:—a heart rate sensor (10) designed to measure the driver's heart rate over time, and—a processing unit (40) designed to a) receive an indication (D) of the danger present in the environment of the vehicle, b) analyse the driver's heart rate measured by the heart rate sensor, during a time interval starting when the indication is received by the processing unit, and c) determine whether or not the danger has been perceived by the driver depending on whether or not the driver's heart rate analysed in step b) comprises a deceleration phase during the time interval.

The present invention relates to the technical field of driving assistance in a vehicle. It relates more specifically to a device for analyzing a vehicle driver's perception of a danger and to an associated method.

At present, it is difficult to know if a driver has perceived a danger present in their surroundings. The driver may, for example, have looked in the direction of a danger without having realized that it is a danger, or, conversely, having perceived a danger only having seen it in their peripheral field of view, but without having looked at it directly. It is also possible that the driver has not seen a danger at all, for example because it is outside of their field of view.

Driving assistance devices that are installed on board vehicles are already known. Such driving assistance devices are capable of analyzing the surroundings of the vehicle on board which they are installed and of intervening with the driver, generally in the form of a visual, audible, haptic and/or olfactory alert, so as to warn them that a danger is liable to come into the range or the path of their driving.

The known driving assistance devices send numerous alerts without any guarantee that those alerts will reach the driver. However, not knowing if a driver has correctly perceived a danger means it cannot be known if the driver is preparing themselves to properly react to this danger, and prevents the design of suitable safety solutions.

Conversely, when the alerts from the driving assistance device reach the driver, the latter may consider them to be disruptive or annoying, notably if they have already perceived the danger associated with the alert, for example because they have already been warned of this danger, or because they have already seen and analyzed this danger themselves. The driver therefore often prefers to deactivate the alerts from the driving assistance device, even if it means running the risk of not being warned of a danger they might not see.

It therefore becomes necessary to make driving assistance devices more relevant, in order for them to be better accepted by drivers, for example by warning the driver only when it is actually useful and by ensuring that the alert has indeed reached the driver.

In this context, the present invention proposes a device for analyzing a vehicle driver's perception of a danger, which device comprises:
a heart rate sensor designed to record the driver's heart rate over time, and
a processing unit designed to:
a) receive an indication of the danger present in the surroundings of the vehicle,
b) analyze the driver's heart rate that was measured by the heart rate sensor, for a time interval which starts upon reception of the indication by the processing unit, and
c) determine if the danger has been perceived or not by the driver depending on whether the driver's heart rate analyzed in step b) comprises or does not comprise a deceleration phase within said time interval.

As a result, the device according to the invention makes it possible to determine if the driver is aware of a danger by analyzing the driver's heart rate immediately after the processing unit has been made aware of the danger. More specifically, the processing unit of the device according to the invention considers that the danger has been perceived by the driver when their heart rate comprises a deceleration phase during the time interval in which analysis takes place, for example in the 3 seconds following the occurrence of the danger. The deceleration phase may extend over the entire time interval (that is to say here over 3 seconds or more) or only over some of the time interval (that is to say here over a duration of less than 3 seconds). Studies have shown that such a cardiac deceleration phase occurs when the heart slows down so that the brain can process information it has received. Depending on the complexity of the information that the brain has received and must process, the duration of the cardiac deceleration phase may be more or less long, the latter being at least partially comprised within a time interval with a duration of between 1 second and 5 seconds, notably a duration of 3 seconds after the information has been received by the brain.

The determination of the driver's perception of the danger by virtue of the device according to the invention is very reliable and fast and does not disrupt or annoy the driver.

Other non-limiting and advantageous features of the device according to the invention, taken individually or in any technically possible combination, are as follows:
in step b), the heart rate analyzed is that of the time interval in which the measurement is taken, having a duration of between 1 second and 5 seconds, preferably between 1 second and 3 seconds, starting from the reception of the indication;
the device comprises an eye sensor designed to detect the direction in which the driver is looking, and the processing unit determines in step c) that the danger has been perceived by the driver if the driver's heart rate analyzed in step b) comprises a deceleration phase and if, furthermore, the driver is looking at the field of driving;
the device comprises a respiration sensor for sensing the driver's breathing that is designed to measure the driver's respiratory rate, and the processing unit is designed to correct the driver's heart rate analyzed in step b) depending on said measured respiratory rate;
the processing unit determines that the driver's heart rate comprises a deceleration phase when the analysis of the heart rate in step b) shows that the variation in the driver's heart rate at an instant t has a lower value than the mean of the values adopted by the variation in heart rate (ΔFC) at the two instants preceding the instant t;
the variation in the driver's heart rate is calculated from a reference heart rate, selected from: the driver's heart rate measured by the heart rate sensor at the instant when the indication is received by the processing unit in step a) and the driver's mean heart rate measured by the heart rate sensor, the driver's mean heart rate preferably being measured when no danger at all is perceived.

According to a particularly advantageous feature of the invention, the processing unit of the device according to the invention is designed to implement a step d) during which it commands a driving assistance device installed on board the vehicle to intervene with the driver, depending on at least one of the following elements: the driver's perception or lack of perception of the danger determined in step c), the moment the danger was perceived by the driver, the duration of the possible phase of deceleration of the heart rate analyzed in step b), the amplitude of the possible phase of deceleration of the heart rate analyzed in step b). Of course, to command the driving assistance device to intervene, the processing unit 40 is designed to take account, in addition to at least one of the aforementioned elements, of the indication of the danger received in step a), in particular of the distance of the danger from the vehicle, and of the degree of severity of said danger.

The fact that the processing unit “commands the driving assistance device to intervene” should be understood to mean that it controls if said driving assistance device should intervene with the driver or not, when it should intervene with the driver, and how (in what form) it should intervene with the driver. Notably, the driving assistance device intervenes with the driver concerning a danger if the driver is not already aware of the danger, in which case the intervention takes place at the best time and in the most suitable form possible to avoid the danger. The device according to the invention is therefore very useful to the driving assistance device installed on board the vehicle.

The interventions that the driving assistance device makes with the driver comprise, as options, the driving assistance device sending a signal to the driver, the signal possibly taking the form of an alert aiming to warn the driver of a danger or the form of a piece of advice aiming to guide the driver in the face of the danger, or the driving assistance device forcibly taking charge of the vehicle, for example by imposing on the driver a switchover to an autonomous driving mode of the vehicle, or a switchover to remotely assisted driving of the vehicle.

For example, if the device according to the invention determines that the driver has not perceived a danger, the processing unit commands that the driving assistance device must intervene with the driver, and controls if the intervention should take the form of an alert sent to warn the driver of this danger, and/or advice sent to guide the driver in the face of the danger, and/or the form of driving assistance imposed on the driver. When the driving assistance is imposed on the driver by the driving assistance device, the driving assistance device is considered to take charge of the vehicle insofar as the members of the vehicle are then directly actuated by the driving assistance device to avoid the danger. Conversely, if the device according to the invention determines that the driver is already aware of a danger, the driving assistance device silences the alerts associated with this danger, such that the driver is not annoyed by the sending of unnecessary alerts.

Other non-limiting and advantageous features of the device according to the invention, taken individually or in any technically possible combination, are as follows:
the device according to the invention comprises the driving assistance device, and the indication of the danger received by said processing unit in step a) is sent by said driving assistance device;
the processing unit is moreover designed to identify the driver and to customize, depending on said identification, the analysis of the heart rate implemented in step b);
the processing unit is moreover designed to customize the command in step d) depending on said identification.

The invention lastly relates to a method for analyzing a vehicle driver's perception of a danger, according to which provision is made to implement the following steps:
measuring the driver's heart rate,
receiving an indication of the danger present in the surroundings of the vehicle,
analyzing the driver's heart rate that is measured, for a time interval which starts upon reception of the indication of the danger, and
determining if the danger has been perceived or not by the driver depending on whether the driver's heart rate that was analyzed comprises or does not comprise a deceleration phase within said time interval.

In addition, various other features of the invention will become apparent from the accompanying description, which is given with reference to the drawings, which illustrate non-limiting embodiments of the invention and in which:

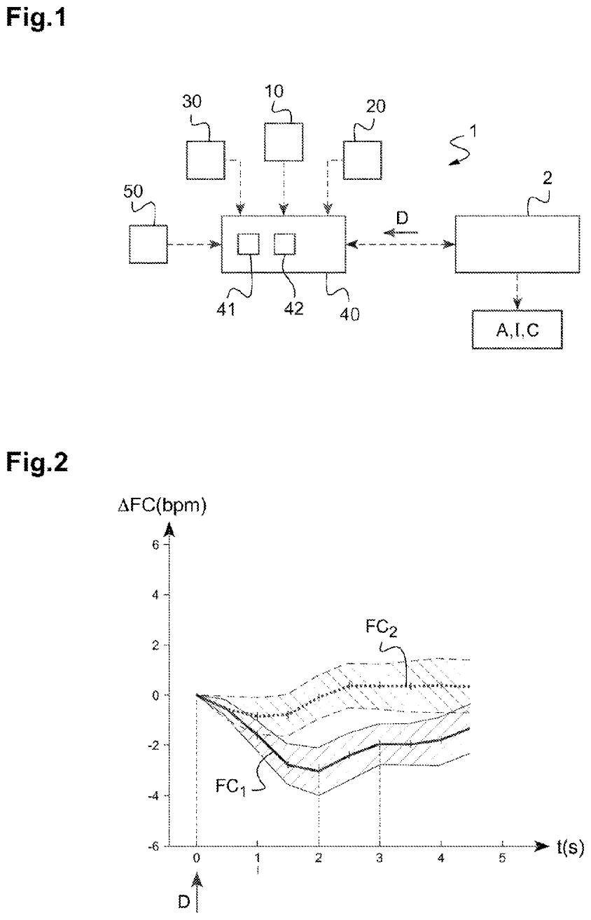

FIG. 1 is a schematic illustration of a device for analyzing the perception of a danger, according to the invention,

FIG. 2 is a graph illustrating two curves FC1 and FC2 giving the change to the variation in heart rate ΔFC (in beats per minute, or bpm) over time (in seconds or s), for one and the same driver, in two different driving situations,

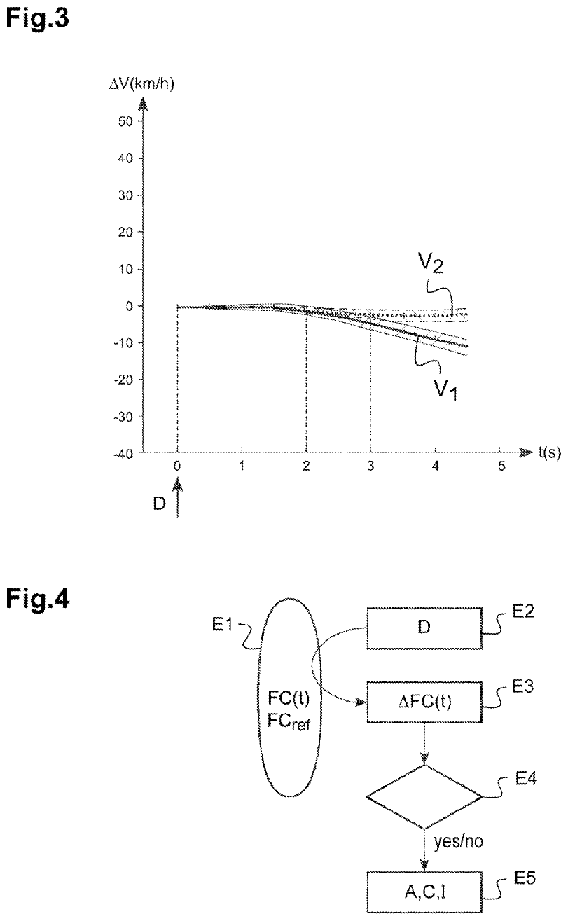

FIG. 3 is a graph illustrating two curves V1 and V2 giving the change to the variation in speed of the vehicle (in kilometers per hour or km/h) over time (in seconds or s), for the driver in the two situations of FIG. 2, and

FIG. 4 is a schematic illustration of the main steps of a method according to the invention.

It should be noted that in these figures the structural and/or functional elements shared by different variants can have the same references.

FIG. 1 schematically illustrates the main elements of a device 1 for analyzing a vehicle driver's perception of a danger. The device 1 is particularly suitable for analyzing the perception of danger in a motor vehicle. The remainder of the description will describe this scenario in detail. It is, however, entirely conceivable for the device 1 according to the invention to be used to analyze the perception of a danger in another type of vehicle, for example a boat, an aircraft or a train.

In this case, the device 1 is installed on board the motor vehicle. It is, however, entirely conceivable for at least some of the elements it comprises to be remote from the vehicle.

The device 1 comprises at least:
a heart rate sensor 10 designed to measure the driver's heart rate over time, and
a processing unit 40 designed to:
a) receive an indication of the danger present in the surroundings of the vehicle,
b) analyze the driver's heart rate measured by the heart rate sensor 10, for a time interval which starts upon reception of the indication by the processing unit 40 in step a), and
c) determine if the danger has been perceived or not by the driver depending on whether the driver's heart rate analyzed in step b) comprises or does not comprise a deceleration phase within said time interval.

The sensor 10 for sensing the driver's heart rate is installed on board the vehicle. It comprises a sensor for sensing the electrical activity of the heart and/or a sensor for sensing the mechanical activity of the heart.

According to a first example, the heart rate sensor 10 has an electrical activity sensor in the form of one or more electrodes, placed directly in contact with the driver's skin, for recording the electrical signal generated by the cardiac activity. In this first example, the heart rate sensor 10 is for example placed on the steering wheel where the driver puts their hands on the steering wheel.

According to another example, the heart rate sensor 10 has a mechanical activity sensor in the form of a blood flow camera or a radar capable of recording the ballistic signal of the driver's cardiac activity, the ballistic signal being directly linked to the mechanical activity generated by the heart beats. In this second example, the heart rate sensor 10 measures the heart rate through the driver's clothing without needing to be in contact with said driver. The heart rate sensor 10 may for example be placed in the driver's seat, behind the steering wheel (that is to say between the steering wheel and the windshield of the vehicle), in the driver's seatbelt, or a combination of these locations.

According to a third example, the heart rate sensor 10 has both an electrical activity sensor and a mechanical activity sensor (ballistic activity in this case), which are for example disposed at one or more of the locations mentioned above.

Irrespective of the example envisaged, the heart rate sensor 10 operates continuously to record the driver's heart rate FC over time while the driver is making journeys. It measures the instantaneous heart rate, that is to say the number of heart beats in a minute (given in beats per minute or bpm). Here, the heart rate sensor 10 gives the driver's instantaneous heart rate FC(t) at regular intervals, for example every 0.5 seconds (s). Of course, it may give the instantaneous heart rate at shorter, and possibly irregular, intervals. Before being able to give its first measurement, it is preferable for the heart rate sensor 10 to have operated for at least one minute.

In practice, to be able to give the driver's instantaneous heart rate, the heart frequency sensor 10 implements the following steps:
it retrieves the duration between two heart beats (or heart beat pair) coming from the driver for a plurality of heart beat pairs,
on the basis of said durations and the number of heart beats measured, it establishes a heart rate for the driver, and
it performs an interpolation to obtain the driver's heart rate every 0.5 seconds.

The processing unit 40 for its part is designed to communicate with the heart rate sensor 10. In this case, it has a memory 41 for storing, as a function of time, the driver's heart rate measured by the heart rate sensor 10. More specifically, the memory 41 stores the driver's instantaneous heart rate FC(t) upon each measurement taken by the heart rate sensor 10, specifically in this example every 0.5 seconds (s).

In step a), the processing unit 40 receives the indication according to which a danger is present in the surroundings of the vehicle. This indication comes from a driving assistance device 2 which is installed on board the motor vehicle and with which the processing unit 40 is designed to communicate. Here, the driving assistance device 2 is considered to be contained in the device 1 according to the invention. It is, however, entirely conceivable for the driving assistance device to be separate from the device 1 according to the invention while still being designed to communicate with the processing unit 40 of the device 1.

The driving assistance device 2 is known per se and will not be described in detail. Essentially, the driving assistance device 2 comprises a plurality of sensors disposed in the vehicle and/or on the outside of the latter such that it is capable of probing the surroundings of the vehicle, whether close by or further away, to determine dangerous situations (or dangers) in these surroundings, for example a pedestrian likely to pass in front of the vehicle, a vehicle traveling the wrong way, sudden braking of another vehicle in front of the vehicle, an accident in front of the vehicle, etc.

The danger detected by the driving assistance device 2 is in this case defined as a function of its severity and/or its distance from the vehicle. The indication D therefore comprises information about the severity (or degree of danger) and/or the distance of the danger from the vehicle.

The driving assistance device 2 is in particular designed to send, to the processing unit 40, the indication D, according to which it has determined that a danger is present in the surroundings of the vehicle as soon as it determines said danger. When the driving assistance device sends such an indication D to the processing unit 40 (step a), the processing unit 40 determines that the instant when it received the indication D from the driving assistance device 2 corresponds to the initial instant, or the instant t=0 s, for the analysis of step b).

As shown in FIG. 1, the driving assistance device 2 is also capable of intervening with the driver, notably by sending the driver an alert A to warn the driver of the danger. This alert A may be sent in various forms: audible (for example a beep or oral notification), visual (for example a message displayed on the dashboard), haptic (for example a vibration felt in the steering wheel or in the seat), olfactory (for example a burning smell released into the passenger compartment), or using a combination of these various forms.

In step b), the processing unit 40 analyzes the driver's heart rate that was measured by the heart rate sensor 10 for a time interval starting at the initial instant (t=0 s) of reception of the indication D. Preferably, the heart rate FC analyzed in step b) is that measured in a time interval having a duration of between 1 second and 5 seconds, even more preferably for a duration of between 1 second and 3 seconds, starting from the reception of the indication D. For example, the time interval starts upon reception of the indication D of danger and extends to 3 seconds after said reception of the indication D.

To perform this analysis, the processing unit 40 in this case comprises a calculator 42 designed to execute said heart rate analysis. This analysis notably comprises the calculation of the variation in the driver's heart rate ΔFC(t) at a given instant, according to the following formula:

ΔFC(t)=FC(t)−FCréf

where FC(t) is the driver's instantaneous heart rate measured by the heart rate sensor 10 at the instant t, and FCréf is a reference heart rate associated with the driver.

In this case, the reference heart rate FCréf associated with the driver is selected from the following list:
the driver's instantaneous heart rate FC(t=0 s) measured by the heart rate sensor 10 at the instant t=0 s when the indication D is received by the processing unit 40 in step a), and
the driver's mean heart rate.
   Preferably, the reference heart rate FCréf associated with the driver is selected as the driver's instantaneous heart rate FC(t=0 s) measured by the heart rate sensor 10 at the instant t=0 s. When it is selected as the driver's mean heart rate, the reference heart rate FCréf is considered to be the driver's specific mean heart rate which is calculated by the calculator 42 from the measurement of the driver's heart rate over a period of at least 1 minute, preferably without any danger at all in the surroundings. The driver's mean heart rate is then preferably recalculated and re-stored regularly in the memory 41 of the processing unit 10. It is also entirely conceivable to consider that the driver's mean heart rate is the driver's resting heart rate. In this case, the mean heart rate originates from a phase of initializing the device 1 according to the invention, during which the driver's heart rate is recorded with the vehicle at a standstill.

In practice, the calculator 42 calculates the value ΔFC(t) adopted by the variation in heart rate at each of the instants t following the initial instant (t=0 s) when the heart rate sensor 10 measured the instantaneous heart rate FC(t).

From the calculated values, it is possible for example to illustrate the curve giving the change to the variation in heart rate ΔFC over time on a graph. FIG. 2, for example, illustrates a graph with two curves FC1 and FC2 which represent the change to the variation in heart rate ΔFC over time, for one and the same driver, in two different driving situations. In this case, each curve FC1 and FC2 is surrounded by a zone representing the error associated with the value illustrated at the instant t. The error zone is illustrated with hatching for each curve FC1 and FC2. In the first situation, associated with the curve FC1, the processing unit 40 has received, at the initial instant (t=0 s), symbolized by the arrow in FIG. 2, the indication D of a vehicle traveling in the opposite direction in the path of the vehicle driven by the driver. In the second situation, associated with the curve FC2, the processing unit 40 has received, at the initial instant (t=0 s), the indication D of a danger liable to occur in the path of the vehicle driven by the driver.

In step c), the processing unit 40 lastly determines if the driver has perceived or not perceived the danger, depending on the analysis in step b).

More specifically, if the driver's heart rate that was analyzed in step b) comprises a deceleration phase (also referred to as “cardiac deceleration”) within said time interval in which the measurement is taken, preferably within the time interval [0; 3 s] of a duration of 3 seconds, the processing unit 40 determines that the driver has perceived the danger. Conversely, if the driver's heart rate that was analyzed in step b) does not comprise a cardiac deceleration phase within said time interval in which the measurement is taken, preferably [0; 3 s], the processing unit 40 determines that the driver has not perceived the danger.

To determine if the heart rate comprises a cardiac deceleration phase, the processing unit 40 looks at whether the variation in the driver's heart rate ΔFC originating from the analysis in step b) comprises a phase of decreasing over time. More particularly, the calculator 42 compares the successive values adopted by the variation in heart rate ΔFC within the time interval in which the measurement is taken, for example between 0 s and 3 s. The processing unit 40 determines that the variation in the driver's heart rate ΔFC comprises a decreasing phase when the variation in heart rate at an instant t, ΔFC(t), has a value lower than the arithmetic mean of the values adopted by said variation in heart rate at the two instants preceding the instant t. In other words, the calculator 42 calculates, for each measurement instant t, the arithmetic mean MOY of the two values adopted by the variation in heart rate at the two instants (t−1) and (t−2) preceding the instant t according to the following formula:

$$MOY = \frac{Δ FC ( t - 1 ) + Δ FC ( t - 2 )}{2}$$

The calculator 42 then compares the value adopted by the variation in heart rate at the instant t, ΔFC(t), with said mean MOY. If the variation in heart rate at the instant t is less than the mean, that is to say ΔFC(t)<MOY, the processing unit 40 determines that the heart rate comprises a deceleration phase over the three instants t−2, t−1 and t.

As a result, in the example of FIG. 2, the processing unit 40 determines that the curve FC1 giving the change to the variation in heart rate over time comprises a decreasing phase, within the interval extending here between 0 and 2 seconds after the processing unit 40 has received the indication D. The processing unit 40 then concludes that in the first situation, the driver has perceived the danger that the vehicle going the wrong way would represent, in the two seconds following the reception of the indication D by the processing unit 40. The driver's perception may be linked to the fact that the driver has seen the vehicle going the wrong way from far away themselves, or to the fact that the driving assistance device 2 has warned the driver by means of an alert A, at the same time as it has sent the indication D to the processing unit 40.

Conversely, in FIG. 2, the curve FC2 does not comprise a decreasing phase within the measurement interval extending between 0 and 5 seconds after the processing unit 40 has received the indication D. The processing unit 40 concludes that the driver in the second situation has not perceived any danger. It is notably possible that the driver has not seen the danger in this second situation themselves, or that they have not perceived the possible alert A sent by the driving assistance device 2, or else that the driving assistance device 2 has failed to send the alert A.

FIG. 3 illustrates a graph with two curves V1 and V2 which represent the change to the

variation in speed ΔV over time, for the vehicle driven by the driver in the two situations described above. In this case, the curves V1 and V2 are each surrounded by a zone representing the error associated with the value illustrated at the instant t. The error zone is illustrated with hatching for each curve V1 and V2 here. The variation in speed of the vehicle is calculated by the following formula:

ΔV(t)=V(t)−Vréf

in which V(t) is the instantaneous speed of the driver at the instant t, and Vréf is the reference speed of the vehicle, in this case selected as the instantaneous speed of the vehicle at the initial instant (t=0 s).

The conclusions of the processing unit 40 of the device 1 according to the invention as to the driver's perception of the danger are confirmed by the driver's different reactions, which can be seen in FIG. 3, in the two situations. Specifically, after having perceived the danger in the first situation, the driver slows down the vehicle they are driving so as to be wary of the danger, such that the curve V1 slopes downward after the 2 seconds taken by the driver to analyze the information about the danger that their brain has received. Conversely, the curve V2 shows a zero variation in speed over time, this meaning that the driver has kept their speed constant in the second situation. This confirms that the driver has not perceived any danger in this second situation.

The device 1 described above may comprise various advantageous improvements which by all means can be combined with one another.

According to a first conceivable advantageous improvement, the device 1 is capable of measuring the driver's heart rate with increased precision.

To that end, the device 1 according to the first improvement comprises a respiration sensor 20 for sensing the driver's breathing that is designed to measure the driver's respiratory rate over time. The respiration sensor 20 is for example placed facing the driver's seat, behind the steering wheel. It is conceivable that the respiration sensor is in line with the heart rate sensor 10, notably when the heart rate sensor 10 has a sensor for sensing mechanical activity of the heart. The respiration sensor 20 is synchronized with the heart rate sensor 10 so as to measure the driver's respiratory rate at the same instants when the driver's instantaneous heart rate is measured. The respiration sensor 20 is designed to measure the driver's respiratory rate, that is to say the number of “breathing in+breathing out” cycles performed by the driver within a time interval of one minute. Like the heart rate sensor 10, the respiratory rate sensor 20 (or respiratory frequency sensor) should preferably have operated for at least one minute before giving its first measurement.

According to this first improvement, the processing unit 40 is designed to communicate with the respiration sensor 20 to store the driver's respiratory rate over time in the memory 41. More specifically, the memory 41 stores the driver's respiratory rate upon each measurement taken by the respiration sensor 20, specifically in this example every 0.5 seconds (s).

The processing unit 40, by virtue of its calculator 42, is then designed to correct the driver's instantaneous heart rate FC(t) depending on said respiratory rate that is measured. The correction consists in eliminating the signal for heart rate measured by the heart rate sensor 10, the signal for respiratory rate measured by the respiration sensor 20, such that the driver's instantaneous heart rate is not influenced by the driver breathing in and breathing out, which will naturally cause said heart rate to fluctuate.

According to a second advantageous improvement, the device 1 according to the invention comprises a means for avoiding detection of false positives, that is to say avoiding the determination that the driver has perceived a danger when this is not the case.

More specifically, the device 1 according to the invention comprises an eye sensor 30 designed to detect the direction in which the driver is looking. The eye sensor 30 for example takes the form of a camera placed behind the steering wheel. The eye sensor 30 is designed to determine if the driver is looking at the field of driving, or if they are looking at an element associated with driving, or else if they are looking at an ancillary element which diverts the driver from driving. Here, “field of driving” is understood to mean the road that can be seen by the driver when they are driving the vehicle. The “elements associated with driving” comprise, for example: rear-view mirrors, the navigation system or else the dashboard from which information relating to the vehicle can originate (speed, lit indicator lights, etc.). The ancillary elements are considered to comprise all the other elements present in the vehicle which do not participate in driving, for example the driver's mobile telephone, the radio station, the passengers in the vehicle. For example, the eye sensor 30 is designed to detect if the driver's head is lowered to look at their mobile telephone or if they have turned around to observe the passengers on the rear bench seat, or the passenger seated next to them, in which case they are not looking at the field of driving or at an element associated with driving.

The eye sensor 30 allows the processing unit 40 to utilize an additional criterion to determine if the driver has perceived the danger or not: the direction in which the driver is looking. This additional criterion avoids the processing unit 40 determining that the driver has perceived a danger solely because their heart rate has a cardiac deceleration phase within the time interval observed. Specifically, the driver's cardiac deceleration is not necessarily caused by the perception of a danger in the field of driving but may be caused by another event, such as reading an SMS for example, or to worrisome indicator lights lighting up on the dashboard. As a result, by virtue of the eye sensor 30, the processing unit 40 utilizes two criteria to determine that the danger has been perceived by the driver: the fact that their heart rate comprises a deceleration phase and the fact that they are looking at the field of driving. In other words, the processing unit 40 determines in step c) that the danger has been perceived by the driver if the driver's heart rate that was analyzed in step b) comprises a deceleration phase and if, furthermore, the driver is looking at the field of driving. However, as long as the driver's heart rate that was analyzed in step b) does not have a deceleration phase, the processing unit 40 concludes directly that the danger has not been perceived, without having to check the direction in which the driver is looking.

As a result, according to this second advantageous improvement, the device according to the invention becomes more precise in the determination of the driver's perception of the danger, by making it possible to deduce the cause of the modulation of the heart rate, said cause possibly coming for example from the road, or from the passenger compartment (suspect indicator light is lit up) or from a text received on the driver's mobile telephone.

According to a third advantageous improvement, the processing unit 40 of the device 1 is designed to command the driving assistance device 2, in particular control if it should intervene with the driver, when it should intervene with the driver and how it should intervene with the driver. By virtue of this command, the driving assistance device 2 disturbs the driver less when it intervenes with them, and the possible interventions with the driver made by the driving assistance device 2 are more relevant because they are made at the right time and in a suitable form.

As mentioned above, the driving assistance device 2 in this case is capable of intervening with the driver, for example by sending alerts A (with audible, visual, haptic and/or olfactory forms) to the driver in order to warn them of a danger that the driving assistance device might have detected in the surroundings of the vehicle. It is possible for the forms in which each alert A is sent to be combined or not combined. The sending of an alert A may be done once or repeated over time, for example at regular time intervals.

In this instance, the driving assistance device 2 is also capable of intervening with the driver by sending the driver advice C to assist them in reacting to a danger. Such advice C sent by the driving assistance device 2 are for example as follows: requesting that the driver slow down, that they return to a specific traffic lane, that they pull over, etc. The advice C that is intended to guide the driver so that they react optimally to a danger is generally sent in a visual form (display on the dashboard) or audible form (advice spoken in the passenger compartment). It is possible for the forms in which each piece of advice C is sent to be combined or not combined. The sending of advice C may be done once or repeated over time, for example at regular time intervals.

The driving assistance device 2 is also capable here of intervening with the driver by imposing a driving aid I on them. Such a driving aid I may consist in relieving the driver of driving, for example by imposing remote-assistance driving on them or by forcing them to switch over to an autonomous driving mode of the vehicle. As a result, the driving aids imposed by the driving device 2 take charge of the members of the vehicle and actuate them in place of the driver.

In this context, the processing unit 40 of the device 1 according to the invention is designed to implement a step d), during which it commands the driving assistance device 2 to intervene with the driver. In particular, the processing unit 40 controls if the driving assistance device 2 should send or resend, or not send or resend, an alert A to the driver to warn them of a danger, at what time, in which form and at what frequency, if it should or should not send one or more pieces of driving advice C to guide the driver in the face of the danger, at what time, in which form and at what frequency, and/or if it should or should not impose a driving aid I on the driver to avoid the danger and at what time.

More specifically, the processing unit 40 implements the command of step d) depending on at least one of the following elements: the driver's perception or lack of perception of the danger determined in step c), the moment the danger was perceived by the driver, the duration of the possible phase of deceleration of the heart rate analyzed in step b), the amplitude of the possible phase of deceleration of the heart rate analyzed in step b).

The analysis of the heart rate in step b) allows the processing unit 40 to know not only if the driver has perceived the danger or not, but also if they have perceived it prematurely, that is to say if the cardiac deceleration has finished right at the start of the interval in which analysis is performed, for example over the first two seconds of the interval [0-3 s], or belatedly, that is to say if the cardiac deceleration started right at the end of the interval in which analysis is performed, for example over the last half-second of the interval [0-3 s]. By virtue of the analysis of the heart rate in step b), the processing unit 40 can thus determine the best time for the driving assistance device 2 to interact with the driver. For example, the best time is determined by the processing unit 40 depending on the rate of decrease in the variation in heart rate in the decreasing phase. More specifically, if the rate of decrease is greater than a predetermined threshold value (that is to say that the rate of decrease is rapid), the processing unit 40 commands the driving assistance device 2 not to intervene, or to silence its alerts. Conversely, if the rate of decrease is less than said predetermined threshold value (that is to say that the rate of decrease is slow), the processing unit 40 commands the driving assistance device 2 to intervene in order to alert and/or advise the driver, or even to intervene with the driving. In practice, if the processing unit 40 does not reveal any cardiac deceleration over the time interval in which analysis is performed, in this instance [0; 3 s], after the indication D of danger has been received, it commands the driving assistance device 2 to intervene with the driver. In other words, if the processing unit 40 analyzes that the cardiac deceleration starts belatedly, that is to say 3 seconds or more after the indication D of danger has been received, or that the cardiac deceleration ends more than 3 seconds after this indication D has been received, it commands the driving assistance device 2 to intervene with the driver in order to alert and/or assist them.

The analysis of the heart rate in step b) moreover allows the processing unit 40 to know if the perception of the danger has worried the driver a little or a lot. The driver's worry is significant when the deceleration lasts a long time and/or when the variation in heart rate is great, that is to say when it drops well below a predetermined threshold value. By virtue of the analysis of the heart rate in step b), the processing unit 40 can therefore determine if the danger has been perceived in all its severity or not, this allowing the processing unit 40 to refine the control of the driving assistance device 2.

Of course, to control the driving assistance device to intervene, the processing unit 40 is designed to take account, in addition to at least one of the aforementioned elements, of the indication D of the danger received in step a), in particular of the distance of the danger from the vehicle, and of the degree of severity of said danger.

The processing unit 40 of the device 1 according to the third improvement is thus designed to receive as input various information relating to the danger and to the way in which this danger has been perceived by the driver, and to send, as output, a command which indicates how the driving device 2 should intervene with the driver. The processing unit 40 thus ensures that the driving assistance device 2 intervenes at the right time and in the form best suited to enable the driver to adopt a suitable behavioral reaction.

According to a fourth advantageous improvement of the device 1 according to the invention, the device 1 is capable of identifying the driver so as to provide customized analysis of the heart rate in step b), and/or customized control of the driving device 2 in step d).

More specifically, the device 1 according to the invention comprises an identification system 50, for example a facial recognition system, or a fingerprint recognition system. The processing unit 40 is designed to communicate with this identification system 50 so as to identify the driver and to retrieve the information it has about this driver in memory.

The processing unit 40 is then designed to customize, depending on said identification, the analysis of the heart rate implemented in step b) and/or the command in step d).

By virtue of the identification system 50, the interventions made by the driving assistance device 2 are best suited to the driver since it is possible to distinguish a novice driver from an experienced driver. Moreover, depending on the driver's sensitivity to certain forms of alerts A rather than others, the driving assistance device 2 favors sending alerts A in the preferred forms. For example, the driving assistance device does not send visual alerts to a far-sighted driver. It sends alerts in combined forms, with a certain urgency, to an elderly person, whereas it sends alerts in a single form to an experienced driver.

According to the same principle, by virtue of the identification system 50, the advice C sent by the driving assistance device 2 is more or less prescriptive, and the form in which it is sent is also adapted to the driver. The driving aids I are also triggered more or less rapidly depending on the driver at the steering wheel.

Moreover, by virtue of the identification system 50, the parameters that define the cardiac deceleration (threshold value and duration) are adapted depending on the driver identified, so as to increase the precision of the analysis in step b) by the processing unit 40.

As a result, by virtue of the device 1 according to the invention, with or without an improvement, it is known if the driver has correctly perceived a danger, such that it is possible to design suitable safety solutions. Moreover, by virtue of the device according to the invention, the driving assistance device 2 intervenes with the driver depending on the danger but also depending on the driver at the steering wheel and the cardiac reactions of this driver, such that the passenger compartment is not polluted by alerts or pieces of advice that are untimely or unsuitable for the situation and the driver. The driving assistance device 2 is thus better tolerated by the driver.

The invention also relates to a method for analyzing the vehicle driver's perception of a danger, as illustrated in FIG. 4.

According to the method of the invention, provision is made to implement the following steps:
measuring the driver's heart rate FC (represented by the block E1 in FIG. 4),
receiving the indication D of the danger present in the surroundings of the vehicle (represented by the block E2 in FIG. 4),
analyzing the driver's heart rate FC that was measured, for a time interval which starts upon reception of the indication D (represented by the block E3 in FIG. 4), and
determining if the danger has been perceived or not by the driver depending on whether the driver's heart rate that was analyzed comprises or does not comprise a deceleration phase within said time interval (represented by the block E4 in FIG. 4).

The device 1 according to the invention is for example designed to implement the method according to the invention. The step of receiving the indication D corresponds to the step a) described above, the step of analyzing the driver's heart rate corresponds to the step b) described above, and the step of determining whether the danger has been perceived or not by the driver corresponds to the step c) described above.

As shown in FIG. 4, upon reception of the indication D (block E2 in FIG. 4) by the processing unit 40 of the device 1, the measurements of the driver's instantaneous heart rate FC(t) that are taken continuously on the driver (block E1 in FIG. 4) are collected to implement the analysis of the driver's heart rate (block E3 in FIG. 4). To do this, the processing unit 40 establishes the instant when the indication D was received as the initial instant t=0 s, and calculates the variation in heart rate ΔFC(t) from the driver's instantaneous heart rate FC(t) and from the reference heart rate FCréf. Depending on this analysis, the processing unit 40 determines if the driver has perceived the danger or not (block E4 in FIG. 4).

Once this determination has been made, the processing unit 40 may optionally (this is illustrated in dashed line in FIG. 4) implement the step d) described above, which is represented by the block E5 in FIG. 4. As a result, the processing unit 40 may possibly command the driving assistance device 2 to intervene with the driver (block E5 in FIG. 4). This optional step (block E5) corresponds to the step d) described above. The command of the interventions A, C, I by the processing unit 40 comprises both sending the alerts A and/or advice C and not sending said alerts A and/or advice (for example by silencing them) or imposing or not imposing a driving aid I on the driver. This optional step (block E5) is implemented both if the processing unit 40 concludes in the determination step (block E4) that the driver has perceived the danger (YES) and if it concludes that they have not perceived the danger (NO).

Of course, various other modifications may be made to the invention within the scope of the appended claims.

1. A device for analyzing a vehicle driver's perception of a danger, the device comprising:
a heart rate sensor configured to measure the driver's heart rate (FC) over time (t), and
a processing unit configured to:
a) receive an indication of the danger present in the surroundings of the vehicle,
b) analyze the driver's heart rate that was measured by the heart rate sensor, for a time interval which starts upon reception of the indication by the processing unit, and
c) determine if the danger has been perceived or not by the driver depending on whether the driver's heart rate that was analyzed in step b) comprises a deceleration phase within said time interval.

2. The device as claimed in claim 1, wherein, in step b), the heart rate analyzed is that of the time interval in which the measurement is taken, having a duration of between 1 second and 5 seconds, starting from the reception of the indication.

3. The device as claimed in claim 1, further comprising: an eye sensor configured to detect the direction in which the driver is looking, and wherein the processing unit determines in step c) that the danger has been perceived by the driver if the driver's heart rate that was analyzed in step b) comprises a deceleration phase and if, furthermore, the driver is looking at the field of driving.

4. The device as claimed in claim 1, further comprising: a respiration sensor for sensing the driver's breathing that is designed to measure the driver's respiratory rate, and wherein the processing unit is configured to correct the driver's heart rate that was analyzed in step b) depending on said measured respiratory rate.

5. The device as claimed in claim 1, wherein the processing unit determines that the driver's heart rate comprises a deceleration phase when the analysis of the heart rate in step b) shows that the variation in the driver's heart rate at an instant t has a lower value than the mean of the values adopted by the variation in heart rate at the two instants preceding the instant t.

6. The device as claimed in claim 5, wherein the variation in the driver's heart rate is calculated from a reference heart rate, selected from: the driver's heart rate measured by the heart rate sensor at the instant when the indication is received by the processing unit in step a) and the driver's mean heart rate measured by the heart rate sensor.

7. The device as claimed in claim 1, wherein the heart rate sensor comprises a sensor for sensing the electrical activity of the heart or a sensor for sensing the mechanical activity of the heart, and wherein said heart rate sensor is disposed on the steering wheel, and/or on the driver's seat and/or on the driver's seatbelt and/or remote from the driver.

8. The device as claimed in claim 1, wherein said processing unit is further configured to:
d) command a driving assistance device installed on board the vehicle to intervene with the driver, depending on at least one of the following elements: the driver's perception or lack of perception of the danger determined in step c), the moment the danger was perceived by the driver, the duration of the possible phase of deceleration of the heart rate analyzed in step b), the amplitude of the possible phase of deceleration of the heart rate analyzed in step b).

9. The device as claimed in claim 8, further comprising the driving assistance device, wherein the indication received by said processing unit in step a) is sent by said driving assistance device.

10. The device as claimed in claim 8, wherein the processing unit is configured to identify the driver and to customize, depending on said identification, the analysis of the heart rate implemented in step b).

11. The device as claimed in claim 10, wherein the processing unit is configured to customize the command in step d) depending on said identification.

12. A method for analyzing a vehicle driver's perception of a danger, comprising:
measuring the driver's heart rate;
receiving an indication of the danger present in the surroundings of the vehicle;
analyzing the driver's heart rate that was measured, for a time interval which starts upon reception of the indication of the danger; and
determining if the danger has been perceived or not by the driver depending on whether the driver's heart rate that was analyzed comprises a deceleration phase within said time interval.
